(19) United States
(12) Patent Application Publication
(10) Pub. No.: US 2016/0004065 A1
(43) Pub. Date: Jan. 7, 2016

(54) Endoscope with Pupil Expander

(71) Applicant: INTEGRATED MEDICAL SYSTEMS INTERNATIONAL, INC., Pembroke Pines, FL (US)

(72) Inventor: Jurgen Zobel, Pembroke Pines, FL (US)

(21) Appl. No.: 14/768,637

(22) PCT Filed: Feb. 19, 2014
(86) PCT No.: PCT/US14/17153

§ 371 Date: Aug. 18, 2015

Related U.S. Application Data
(60) Provisional application No. 61/766,576, filed on Feb. 19, 2013.

(51) Int. Cl. G02B 23/24; A61B 1/05; G02B 23/02; A61B 1/00

(52) U.S. Cl. CPC G02B 23/2415; A61B 1/00193; A61B 1/00096; A61B 1/05; G02B 23/243; G02B 23/02

(57) ABSTRACT

Disclosed are stereo endoscopes where the optical train of one optical system is separated in a left and right optical train. The separation in a left and right optical train is achieved by splitting the entrance pupil in a left and right pupil half. To additionally achieve a significant stereoscopic disparity the distance between the left and right pupil half is expanded. The separation of the entrance pupil and the expansion of the left and right pupil half is achieved by a distally located prism block. This prism block consists of a pair of rhomboidal prisms which are located exactly at the entrance pupil inside the endoscope objective. The front lens group is replaced by an identical pair of front lens groups each assembled in front of one of the two rhomboidal prisms. Such stereo endoscopes with expanded pupil halves enable to build stereo endoscopes with very small diameter or stereo endoscopes for special surgical applications. The described stereo endoscopes also include video endoscopes where the aperture functioning as the entrance pupil is separated in two halves and expanded for better stereoscopic effect. The prism block of the disclosed stereo endoscopes can also be used to separate the exit pupil of the disclosed stereo endoscopes and expand the optical axes on the proximal side to adapt to different stereo endoscopic cameras.

### FIELD OF THE INVENTION

The present invention is directed to an optical system for an endoscope. More particularly, the present invention is directed to an optical system for a monoscopic endoscope, the optical system including a pupil expander assembly for creating two stereoscopic images.

BACKGROUND OF INVENTION

Technical and medical endoscopes are delicate optical instruments that are introduced in technical and human cavities to inspect the interior of the cavities. Such endoscopes can be rigid endoscopes containing a lens system, flexible endoscopes containing a flexible image guiding bundle or video endoscopes.

Endoscopes have a small diameter of a few millimeters and are often several hundreds of millimeter long. Endoscopes contain an outer tube and an inner tube. The space between the outer tube and the inner tube is filled with illumination fibers that guide externally created light inside the cavities. Inside the inner tube is an optical system that relays an image of the cavity from the distal tip of the endoscope back to the proximal end of the endoscope. This relayed image can be observed at the proximal end by the operator's eye, or a video camera can capture the image.

When the first endoscopes with such lens systems where introduced at the beginning of the 20th century surgeons and designers contemplated stereo endoscopic systems. Stereoscopic systems in general produce two images that show an object space from slightly different perspectives, namely, a left image and a right image. The human brain is capable of merging these two slightly different images and transferring the so-called disparity of the two images, via information about depth, and creates a three-dimensional impression.

Endoscopes with a single optical system, i.e., monoscopic endoscopes, create three-dimensional information about the object space. Objects in different distances appear in different sizes, and overlapping of objects informs the user which object is in the front and which is in the back. In this way, endoscopic instruments inserted through and viewed by an endoscope can be observed by the user moving back and forth in the object space. For many surgical applications in endoscopy this three dimensional information is sufficient, and there is no need for stereoscopic vision. However, with an increasing number of complex surgical procedures being performed and observed through endoscopes, the control of the position of endoscopic instruments has become more crucial for some surgeries. Such complex surgeries are now often performed with robotic assistance, and the position of the instruments is controlled by stereo endoscopic observations.

Stereo endoscopes typically include two optical systems that are difficult to assemble and align and expensive to manufacture. For these reasons, stereo endoscopes having two optical systems are not economically or technically feasible for certain applications. In those instances, it is preferred use a monoscopic endoscope that is modified to provide a stereo endoscopic capability by separating the pupil of the single optical system into a left portion and a right portion. A shortcoming of endoscopes of this type of modified endoscope, however, is that the stereoscopic eye base of such endoscopes is about half the entrance pupil of the endoscope. Such entrance pupils have a diameter of a few tenth of a millimeter. This results in a reasonable stereoscopic working distance of only around one mm or less than one mm. The present invention addresses the small stereoscopic eye base problem that is associated with monoscopic endoscopes that have been modified as explained above by providing an endoscope having a single optical system with a pupil expander.

### SUMMARY OF THE INVENTION

The present invention is directed to stereo endoscopes that include a single optical system or train but that have an extended distance between the left image side and the right image side of the entrance pupil. According to the present invention, the optical train of a unitary optical system is separated into a left optical train and a right optical train. The separation into left and right optical trains is achieved by splitting the entrance pupil into a left and right pupil half.

To additionally achieve a significant stereoscopic disparity the distance between the left and right pupil half is expanded. The separation of the entrance pupil and the expansion of the left and right pupil halves are achieved by a distally located prism block. This prism block consists of a pair of rhomboidal prisms that are located exactly at the entrance pupil inside the endoscope objective. A front lens group is assembled in front of each of the two rhomboidal prisms. Such stereo endoscopes with expanded pupil halves enable to build stereo endoscopes with very small diameter or stereo endoscopes for special surgical applications.

The information for the left and right perspective of the endoscopic image is related to the left and right half of the entrance pupil. The circular entrance pupil of such a stereo endoscope can be optically separated, and the distance of the two halves of the entrance pupil can be expanded and the stereoscopic effect can be increased.

According to one aspect of the invention, there is provided a prism block that is located at the surface of the entrance pupil. The prism block includes of a pair of rhomboidal prisms that are located exactly at the entrance pupil inside the endoscope objective. The two rhomboidal prisms touch at one side and divide the entrance pupil in half. The rhomboidal prisms are arranged so that the entrance pupil of the endoscope sits exactly at the exit surface of the rhomboidal prisms. In this manner, one rhomboidal prism deflects all rays going through the left portion of the entrance pupil to the left and transfers this half of the entrance pupil to the left. The other rhomboidal prism deflects all rays going through the right portion of the entrance pupil to the right and transfers this half of the entrance pupil to the right. The deflection angles of the two rhomboidal prisms have a slightly different angle so that the optical axes of the left and right rhomboidal prisms converge in the working distance of the stereo endoscope.

The optical system further includes the two front lens groups that are aligned in front of the left and the right halves of the entrance pupil. The two front lens groups are glued on a wedged glass plate so that the optical axes of the negatives correspond to the converging optical axes exiting the two rhomboidal prisms. The two units, each consisting of one of the negative lens groups and one of the wedged glass plates, are then aligned under optical control so that the optical fields of the two optical trains overlap in the working distance.

According to another aspect of the invention, the two rhomboidal prisms are fixed on a triangle prism, and the combination of these three prisms is glued to a glass window. The glass window and the three prisms form a prism block that can be easily aligned in front of the entrance pupil of the stereo endoscope and fixed to a plano surface of the endoscope objective.

According to another aspect of the invention, at a proximal end of the described stereo endoscopes, the left half of the exit pupil contains the information gathered through the expanded left side of the entrance pupil and the right half of the exit pupil contains the information gathered through the expanded right side of the entrance pupil. This information in the exit pupil of the stereo endoscope can be separated by a prism block including two rhomboidal prisms placed in the tip of a stereo endoscope camera.

According to yet another aspect of the invention, the prism block comprising two rhomboidal prisms can be used to separate the entrance pupil of a video endoscope objective. In this embodiment, the prism block is positioned at the aperture which represents the entrance pupil of the video endoscope objective to create two halves of the aperture. On the video chip the two images from the left and right perspective are overlaid. The two images are alternatingly blocked by a LCD shutter thereby allowing the left and right image to be read out from the chip separately. Such a LCD shutter can be integrated in the prism block by substituting the prior described glass window holding the prisms with a pair of LCD shutters. If the image signal for the left and the right image is then processed separately, the stereo video signal can be displayed with the stereo endoscope system described in WO 2014012103, titled “Stereo Endoscope System”. In particular, the stereo endoscope of the present invention can be mechanically and optically coupled to the stereo video cameras described in WO 2014012103 A1.

The described prism block can also be used to separate the exit pupil of stereo endoscopes in a left and right half and expand the distance between the optical axes of the two halves to adapt the stereo endoscope to the optical axes of any stereo endoscope camera.

### BRIEF DESCRIPTION OF THE DRAWINGS

FIG. 1 is a sectional view of a stereo endoscope optical system including two optical trains in accordance with the prior art.

FIG. 1A is a front view of the stereo endoscope optical system of FIG. 1 depicting a pair of entrance pupils.

FIG. 2 is a sectional view a stereo endoscope optical system including a single optical train in accordance with the prior art.

FIG. 2A is a front view of the stereo endoscope optical system of FIG. 2 depicting a pair of entrance pupils.

FIG. 3 is a sectional view of a stereo endoscope optical system including a single optical train and a pupil expander assembly in accordance with the present invention.

FIG. 3A is a front view of the stereo endoscope optical system of FIG. 3 depicting an entrance pupil of the optical system divided and separated into a left half and a right half.

FIG. 4 is a sectional view of the stereo endoscope optical system of FIG. 3 displaying right perspective and left perspective views of a point in an object field of the optical system.

FIG. 5 depicts a pair of negative front lens, a pair of rhomboidal prisms, the divided entrance pupil and a plano-convex lens of the stereo endoscope optical system of FIG. 3.

FIG. 6 depicts convergent optical axes of the pair of rhomboidal prisms of the stereo endoscope optical system of FIG. 5.

FIG. 7 depicts the pair of rhomboidal prisms of the stereo endoscope optical system of FIG. 5 assembled into a prism block assembly.

FIG. 8 is magnified view of the stereo endoscope optical system of FIG. 3.

FIG. 9 is a sectional view of a stereo endoscope optical system, where the pair of rhomboidal prisms of the stereo endoscope sits in front of a deflecting prism.

### DETAILED DESCRIPTION OF PREFERRED EMBODIMENT AND FIGURES

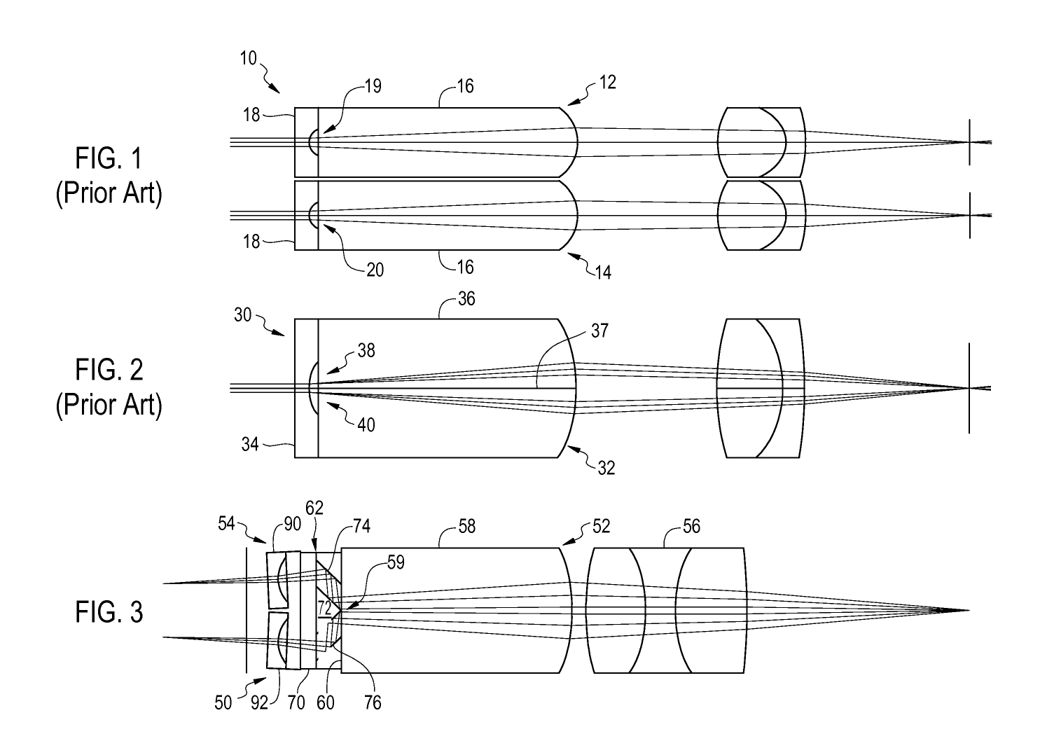

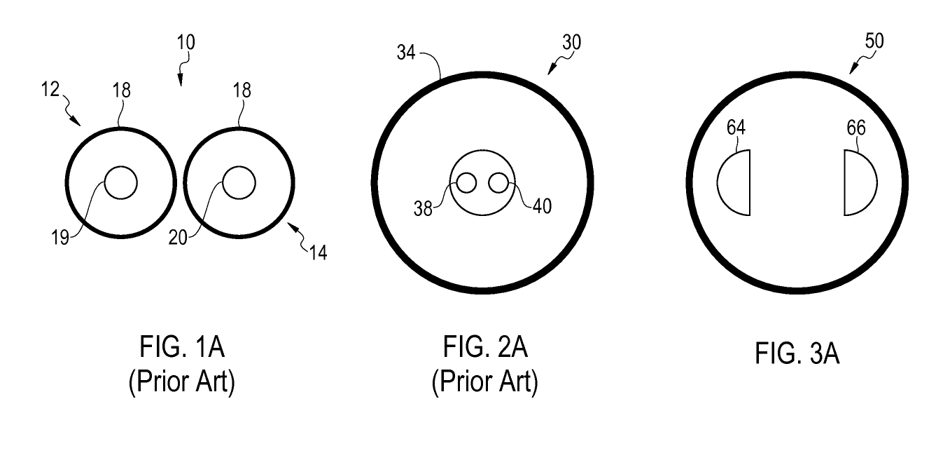

As described above, prior art stereo endoscope optical systems present in two basic configurations including those with two optical systems and those with a single optical system. Referring to FIG. 1, there is depicted a stereo endoscope optical system 10 including two separate optical systems or trains 12, 14. Each of optical systems 12, 14 includes a negative front lens 18 cemented to a plano surface of a plano-convex lens 16 of an objective. As depicted in FIGS. 1 and 1A, use of two separate optical systems 12, 14 provides stereo endoscope optical system 10 with two circular entrance pupils including a left entrance pupil 19 and a right entrance pupil 20 having an extended distance there between. A benefit of stereo endoscope optical system 10 is that it provides adequate right and left perceptive views of the object or working field. A shortcoming of stereo endoscope optical system 10 is that the diameter of the distal end of an endoscope must be great enough to accommodate both optical systems 12, 14.

Referring to FIG. 2, there is depicted a stereo endoscope optical system 30 including a single optical system or train 32. Optical system 32 includes a negative front lens 34 cemented to a plano surface of a plano-convex lens 36 of an objective. As depicted in FIGS. 2 and 2A, a divider 37 functions to provide stereo endoscope optical system 30 with two, relatively small circular entrance pupils including a left entrance pupil 38 and a right entrance pupil 40 having an small distance there between. A benefit of stereo endoscope optical system 30 is that the diameter of the distal end of an endoscope required to accommodate single optical system 32 is smaller than what is required for stereo endoscope optical system 10. A shortcoming of stereo endoscope optical system 30 is that the right and left perceptive views provided of the object or working field of the system are inadequate for some medical and surgical applications because of the proximity of the right and left pupils 38, 40 to one another and the diameter of the pupils.

The present invention relates to a stereo endoscope optical system that overcomes the shortcomings of prior art stereo endoscope optical systems by utilizing a single optical system or train in combination with a pupil expander assembly. The pupil expander assembly divides the single entrance pupil of the single optical train into a left half and a right half and separates the left half from the right by an extended distance. The resulting stereo endoscope optical system can be used in a distal end of a surgical endoscope having a diameter that is as small as the diameter of endoscopes including a conventional, single optical system, while providing perspective right and left views of an object field like what is afforded by endoscope that include two, separate optical systems.

Referring to FIG. 3 there is depicted a stereo endoscope optical system 50 including a single optical train 52 and a pupil expander assembly 54 in accordance with the present invention. Single optical train 52 is defined by relay lens assembly 56 that is optically aligned with an objective including a plano-convex lens 58. Single optical train 52 includes an entrance pupil 59 that is located at a plano surface 60 of the plano-convex objective lens. Supported directly on plano surface 60 is a prism block 62 that functions to separate entrance pupil 59 into a left half 64 and a right half 66 and deflect the light rays sideward.

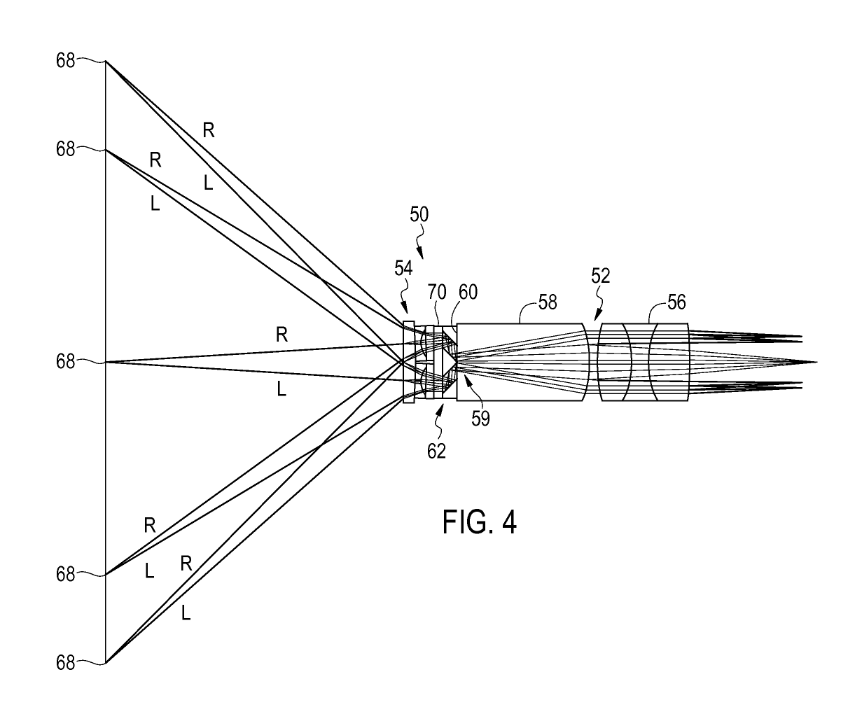

As depicted in FIG. 4, combining single optical train 52 with pupil expander assembly 54 results in a significantly expanded distance between left and right halves 64, 66 of entrance pupil 59, as observed from the object field. Consequently, each object point 68 in the object field is viewed from two perspective points with significant lateral distance. The directions from the left and right perspective points are designated in FIG. 4 with the capital letters L and R.

Referring to FIGS. 3 and 4, the distance between left and right halves 64, 66 of entrance pupil 59 is expanded by pupil expander 54 and primarily by prism block 62. Prism block 62 includes a glass window 70 cemented to a triangle prism 72. On a left side and a right side of triangle prism 72 are two rhomboidal prisms 74, 76 cemented. Left and right sides of rhomboidal prisms 74, 76 are supported in place by support prisms 78, 80 cemented (prisms 78, 80 not shown in FIG. 4 but in FIG. 7).

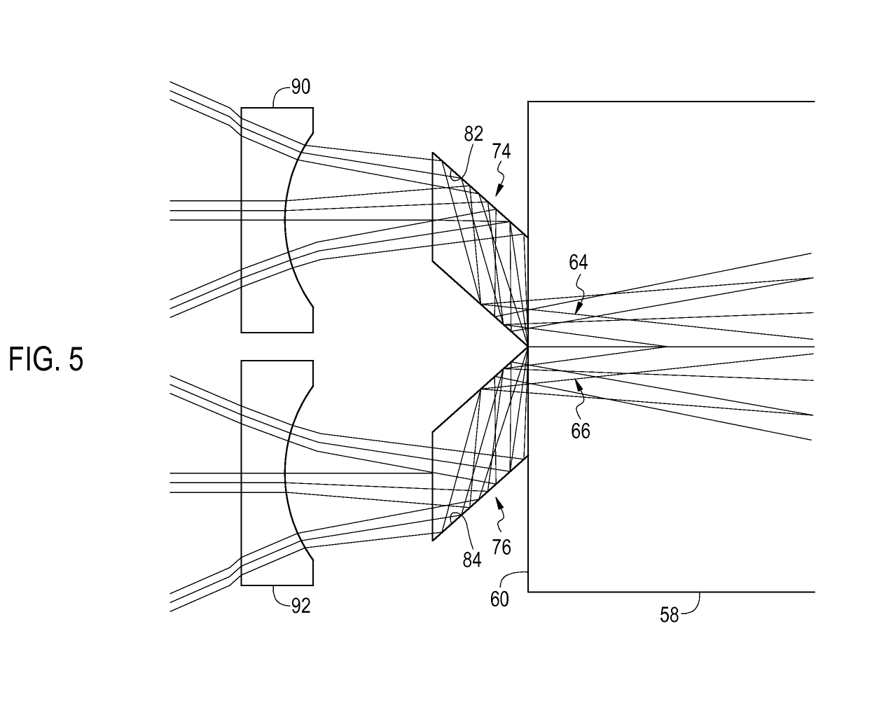

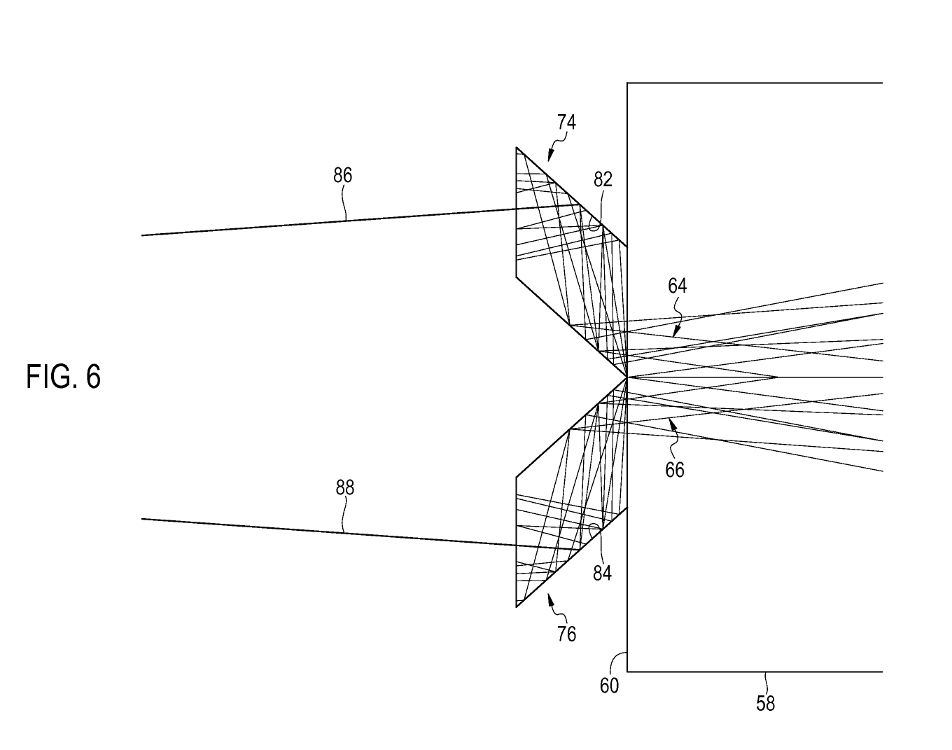

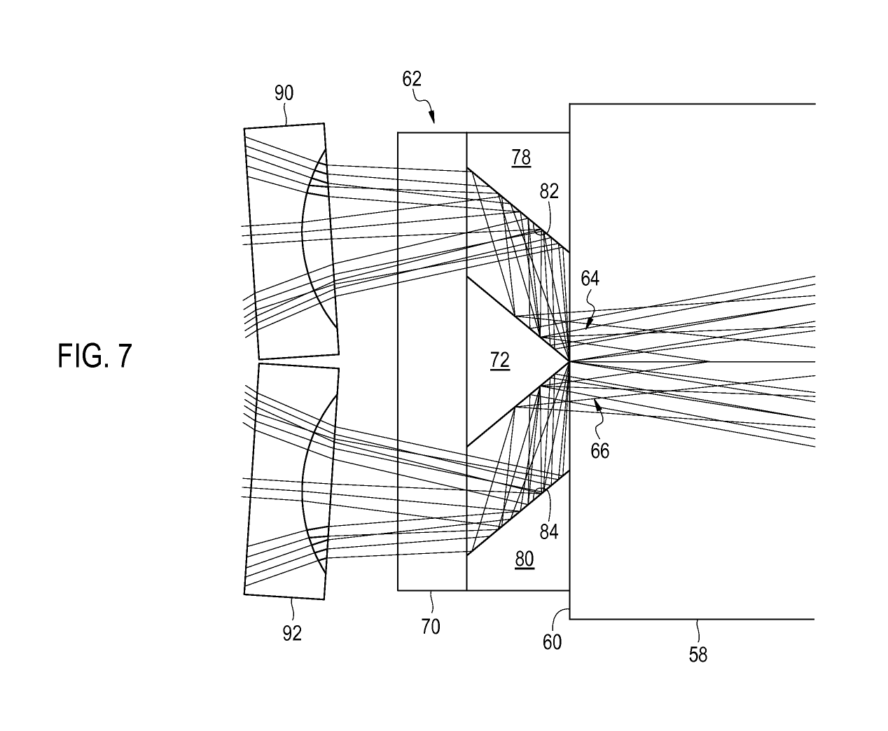

Rhomboidal prisms 74, 76 of prism block 62 divide and separate entrance pupil 59 of the stereo endoscope. As depicted in FIGS. 5 and 6, rhomboidal prisms 74, 76 include respective reflecting sides 82, 84 having slightly different angles which results in the convergence of the two optical axes 86, 88 on the object side.

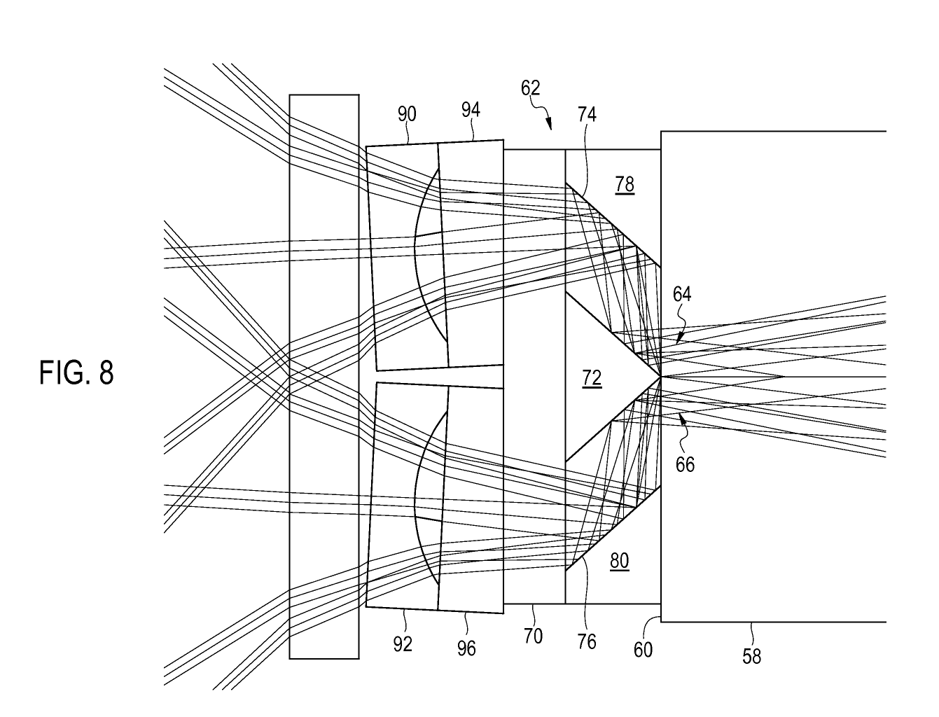

Referring to FIGS. 5 and 7, in front of prism block 62 are located a pair of negative front lenses 90, 92 that are optically aligned with rhomboidal prisms lenses 74, 76, respectively. As depicted in FIG. 8, negative front lenses 90, 92 can be cemented on respective wedged glass plates 94, 96 and aligned in front of and cemented to prism block 62.

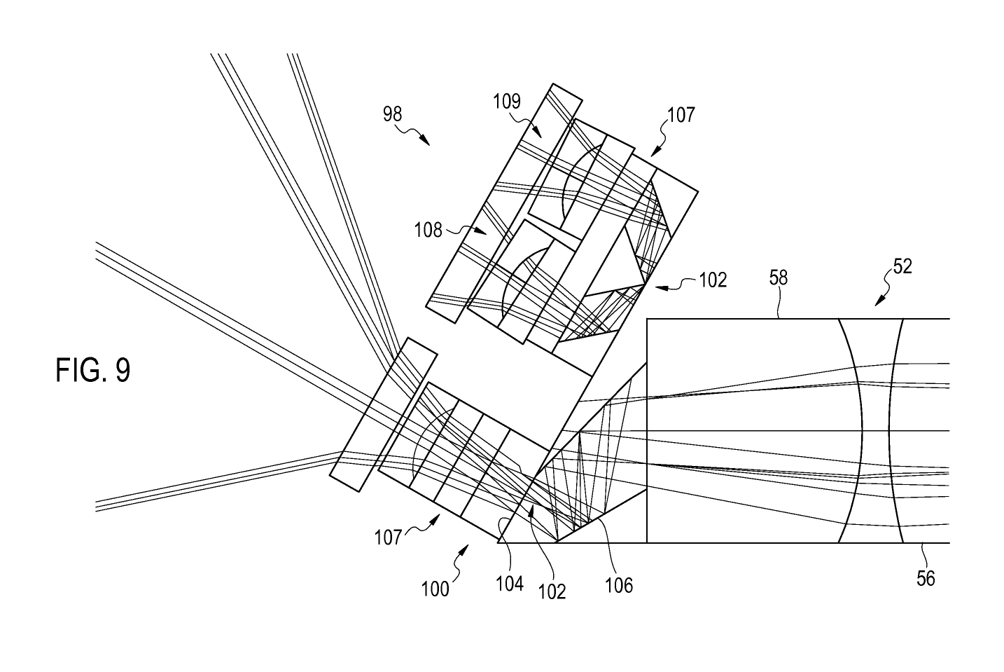

Referring to FIG. 9, there is depicted a stereo endoscope optical system 98 including single optical train 52 and a pupil expander assembly 100 in accordance with the present invention. Single optical train 52 is defined by relay lens assembly 56 that is optically aligned with an objective including plano-convex lens 58. In this embodiment, entrance pupil 102 is located at an exit surface 104 of a deflection prism 106. On exit surface 104 is a prism block 107 cemented which separates entrance pupil 102 into a left portion 108 and a right portion 109 and expands the distance between the two pupil halves.

As will be apparent to one skilled in the art, various modifications can be made within the scope of the aforesaid description. Such modifications being within the ability of one skilled in the art form a part of the present invention and are embraced by the claims below.

It is claimed:

1. An optical system for an endoscope comprising:
an objective lens,
an entrance pupil having an optical axis,
a prism block located adjacent to an entrance pupil side of the objective lens, the prism block including a first rhomboidal prism and a second rhomboidal prism,
wherein the first rhomboidal prism has a first corner and the second rhomboidal prism has a second corner, the first corner and the second corner being in contact at a point located on the optical axis of the entrance pupil.

2. The optical system according to claim 1 wherein the first corner is partially defined by a first side that lies directly against the objective lens and the second corner is partially defined by a second side that lies directly against the objective lens.

3. The optical system according to claim 1 wherein the objective lens is a plano-convex lens, the entrance pupil being located at a plano surface of the plano-convex lens.

4. The optical system according to claim 1 wherein the point is located immediately adjacent to the entrance pupil.

5. The optical system according to claim 1 wherein the prism block includes a triangle shaped prism coupled to and between the first rhomboidal prism and the second rhomboidal prism.

6. The optical system according to claim 5 wherein the prism block includes a first supporting prism coupled to the first rhomboidal prism and a second supporting prism coupled to the second rhomboidal prism.

7. The optical system according to claim 1 further comprising a first negative lens set being optically aligned with the first rhomboidal prism and a second negative lens set being optically aligned with the second rhomboidal prism.

8. The optical system according to claim 7 comprising a first wedged glass plate optically aligned with and coupled to and between the first negative lens set and the first rhomboidal prism and a second wedged glass plate optically aligned with and coupled to and between the second negative lens set and the second rhomboidal prism.

9. The optical system according to claim 1 wherein the first rhomboidal prism has a first reflecting side with a first deflection angle and a second reflecting side with a second deflection angle and the second rhomboidal prism has a third reflecting side with a third deflection angle and a fourth reflecting side a fourth deflecting angle.

10. The optical system according to claim 9 wherein the first rhomboidal prism has an optical axis that converges in a working distance with an optical axis of the second rhomboidal prism.

11. A method of using the optical system of claim 1 comprising dividing the entrance pupil in half thereby creating two stereoscopic images, each of the two stereoscopic images being observed from a right perspective and a left perspective.

12. A stereo endoscope including a monoscopic endoscope having a pupil that is divided into a left half and a right half thereby creating two stereoscopic images that are observed from a left perspective and a right perspective, the stereo endoscope comprising:
a first prism block including a pair of rhomboidal prisms that are located immediately adjacent to an entrance pupil inside an endoscope objective and arranged to divide the entrance pupil in half, and
a pair of negative lens groups optically aligned with the pair of rhomboidal prisms,
wherein a first rhomboidal prism of the first prism block deflects all rays extending through a left half of the entrance pupil to the left and transfers a left half of the entrance pupil to the left and second rhomboidal prism of the first prism block deflects all rays extending through a right half of the entrance pupil to the right and transfers a right half of the entrance pupil to the right.

13. The stereo endoscope according to claim 12 wherein each rhomboidal prism of the pair of rhomboidal prisms includes two reflecting sides having different diffraction angles.

14. The stereo endoscope according to claim 13 wherein the pair of rhomboidal prisms have optical axes that converge in a working distance of the stereo endoscope.

15. The stereo endoscope according to claim 12 wherein the first prism block includes a triangle prism and a glass window fixed to the pair of rhomboidal prisms.

16. The stereo endoscope according to claim 15 wherein the first prism block includes at least one support prism coupled to at least one rhomboidal prism of the pair of rhomboidal prisms.

17. The stereo endoscope according to claim 12 wherein the pair of negative lens groups are glued on wedged glass plated and optically aligned with converging optical axes extending from the pair of rhomboidal prisms.

18. The stereo endoscope according to claim 12 further comprising a second prism block including a pair of rhomboidal prisms that are located immediately adjacent to an exit pupil of the endoscope objective and arranged to divide the exit pupil in half.

19. The stereo endoscope according to claim 18 further comprising a stereo endoscope camera operatively coupled to a proximal end of the endoscope objective.

20. The stereo endoscope according to claim 19 further comprising an LCD shutter arranged between the exit pupil and the stereo endoscope camera and configured for selectively blocking a left half of the exit pupil and a right half of the exit pupil.

21. A method of expanding an endoscope optical system pupil comprising:
providing an monoscopic optical system having an entrance pupil,
using a first rhomboidal prism to deflect substantially all rays extending through a first half of the entrance pupil in a first direction, and
using a second rhomboidal prism to deflect substantially all rays extending through a second half of the entrance pupil in a second direction that is away from the first direction.

22. The method according to claim 21 further comprising arranging the first rhomboidal prism and the second rhomboidal prism to have converging optical axes that converge in a working distance.

23. The method according to claim 21 further comprising optically aligning a first negative lens group with the first rhomboidal prism and optically aligning a second negative lens group with the second rhomboidal prism.

24. The method according to claim 21 further comprising arranging a first corner of the first rhomboidal prism to contact a second corner of the second rhomboidal prism along an optical axis of the entrance pupil.

25. The method according to claim 21 further comprising forming a prism block with the first rhomboidal prism and the second rhomboidal, fixing the prism block to a plano surface of an objective lens wherein the entrance pupil is positioned immediately adjacent to the prism block.

26. The method according to claim 21 further comprising arranging the first rhomboidal prism and the second rhomboidal prism to be immediately adjacent to the entrance pupil.

27. The method according to claim 21 wherein the first rhomboidal prism and the second rhomboidal prism are located at a surface of the entrance pupil.

28. The method according to claim 21 further comprising using the first rhomboidal prism and the second rhomboidal prism to create two stereoscopic images that are observed from a left perspective and a right perspective.

29. The method according to claim 21 further comprising using a third rhomboidal prism to deflect substantially all rays extending through a first half of an exit pupil of the monoscopic optical system and using a fourth rhomboidal prism to deflect substantially all rays extending through a second half of the exit pupil to the right.

30. The method according to claim 29 further comprising operatively coupling the third rhomboidal prism and the fourth rhomboidal prism between a stereo endoscope camera and the exit pupil.
